(19) United States
(12) Patent Application Publication
(10) Pub. No.: US 2026/0069336 A1
(43) Pub. Date: Mar. 12, 2026

(54) PORTABLE VACUUM MIXING DEVICE FOR BONE CEMENT DURING SURGERY

(71) Applicant: The Fourth Medical Center of the Chinese People's Liberation Army General Hospital, Beijing (CN)

(72) Inventors: Licheng ZHANG, Beijing (CN); Xiang CUI, Beijing (CN); Chi MA, Beijing (CN); Houchen LV, Beijing (CN); Yuheng JIANG, Beijing (CN); Hailin XU, Beijing (CN); Chengla YI, Beijing (CN); Qiyong CAO, Beijing (CN); Bin YU, Beijing (CN); Hua CHEN, Beijing (CN); Xuedong BAI, Beijing (CN); Yong XIE, Beijing (CN); Jing ZHANG, Beijing (CN)

(73) Assignee: The Fourth Medical Center of the Chinese People's Liberation Army General Hospital, Beijing (CN)

(21) Appl. No.: 19/394,135

(22) Filed: Nov. 19, 2025

(30) Foreign Application Priority Data
Dec. 18, 2024 (CN) 202411868102.5

(51) Int. Cl. A61B 17/88

(52) U.S. Cl. CPC A61B 17/8833; A61B 17/8822; A61B 2017/8838

(57) ABSTRACT

A portable vacuum mixing device for bone cement during surgery, includes an extraction housing and a mixing housing. A miniature vacuum pump is fixedly connected to an upper end of the extraction housing, a powder raw material inlet is provided at a position of the upper end of the extraction housing close to the miniature vacuum pump, and a liquid raw material storage cavity is formed inside the extraction housing. A mixing chamber is provided inside the mixing housing, and a bone cement mixing unit is provided inside the mixing housing. The device is compact and portable, allowing for its use directly within the operating room during an ongoing surgery. Moreover, by employing the bone cement mixing unit, the device significantly reduces manual labor intensity. Owing to the implementation of vacuum mixing, the device achieves a better mixing effect.

### CROSS-REFERENCE TO RELATED APPLICATIONS

This application claims priority to Chinese Patent Application No. 202411868102.5 with a filing date of Dec. 18, 2024. The content of the aforementioned application, including any intervening amendments thereto, is incorporated herein by reference.

### TECHNICAL FIELD

The present disclosure relates to the field of medical instruments, and in particular, to a portable vacuum mixing device for bone cement during surgery.

### BACKGROUND

Bone cement, as a medical adhesive widely used in orthopedic surgery, is primarily employed for bone fixation, defect filling, and fracture healing. It is an adhesive material capable of tightly bonding two different materials (such as bone and an implant, or bone to bone) and maintaining stability over a period of time.

Polymethyl methacrylate bone cement (PMMA bone cement) is a type of bone cement. The PMMA bone cement is composed of a liquid monomer component and a powder component, and it can be prepared by mixing the cement powder with the monomer liquid in an appropriate mixing cup with the aid of a spatula. However, a drawback of this method is the potential entrapment of air within the resulting cement paste, which can subsequently lead to a loss of mechanical stability in the bone cement. Therefore, the mixing of the bone cement powder and the monomer liquid should preferably be performed in a mixing device equipped with a vacuum source, as this vacuum mixing process minimizes air entrapment in the cement paste, thus achieving optimal cement quality.

As disclosed in Chinese Patent No. CN107008197B, a vacuum mixing device is provided in the prior art. The vacuum mixing device is provided with at least one cylinder, a mixing component, a receiving seat, an opening mechanism, a pump, and connecting piping. The vacuum mixing device is free from external auxiliary means, such as a compressed air-driven vacuum pump and vacuum hose, and can be operated independently of an energy source, such as compressed air or batteries. The vacuum mixing device is a stand-alone unit that can be operated under all surgical conditions, from the simplest to the most challenging.

Although the device in the prior art enables vacuum mixing of bone cement under all surgical conditions, from the simplest to the most challenging, without relying on external auxiliary means such as a compressed air-driven vacuum pump and hose, and an energy source such as compressed air or batteries, thereby achieving a certain expansion of applicable scenarios, the device cannot be employed in a confined space due to its overall bulk and poor portability, for instance, it cannot be deployed within an operating room during an ongoing surgical procedure. Furthermore, the reliance on manual mixing in the prior art not only imposes a substantial physical burden on the operator but also leads to suboptimal mixing outcomes. Additionally, the relatively complex mixing structure results in a cumbersome cleaning process after the bone cement has been mixed, indicating that there is still considerable room for improvement in terms of user convenience.

### SUMMARY

For the problems existing in the prior art, an objective of the present disclosure is to provide a portable vacuum mixing device for bone cement during surgery.

To solve the above problems, the present disclosure adopts the following technical solutions:

A portable vacuum mixing device for bone cement during surgery, including an extraction housing and a mixing housing, where the extraction housing is detachably connected to the mixing housing; a miniature vacuum pump is fixedly connected to an upper end of the extraction housing, a powder raw material inlet is provided at a position of the upper end of the extraction housing close to the miniature vacuum pump, a liquid raw material storage cavity is formed inside the extraction housing, a liquid raw material injection port communicating with the liquid raw material storage cavity is formed in one end of the extraction housing, a liquid raw material extraction rod is slidably and sealingly connected into the liquid raw material storage cavity, a powder injection port and a vacuum suction port are arranged symmetrically about the liquid raw material injection port at one end of the extraction housing, the powder injection port communicates with the powder raw material inlet through a pipe, and the vacuum suction port communicates with the miniature vacuum pump through a pipe; and
a mixing chamber is provided inside the mixing housing, the mixing chamber communicates with the liquid raw material injection port, the powder injection port, and the vacuum suction port, a bone cement discharge port is formed in a middle of one end of the mixing housing, the bone cement discharge port communicates with one end of the mixing chamber, and a bone cement mixing unit is provided inside the mixing housing.

Optionally, a control panel is provided on one side of an upper surface of the extraction housing, and a storage battery is provided at a position of the upper surface of the extraction housing close to the control panel.

Optionally, the mixing chamber includes a trapezoidal interface chamber, three connecting ports are formed in one end of the trapezoidal interface chamber, the three connecting ports are detachably connected to the powder raw material inlet, the powder injection port, and the vacuum suction port, respectively, one end of the trapezoidal interface chamber is fixed to and communicates with a first olive-shaped chamber, one end of the first olive-shaped chamber is fixed to and communicates with a middle converging channel, one end of the middle converging channel is fixed to and communicates with a second olive-shaped chamber, and the second olive-shaped chamber communicates with the bone cement discharge port.

Optionally, the trapezoidal interface chamber, the first olive-shaped chamber, and the second olive-shaped chamber are all made of rubber, and the middle converging channel is made of hard plastic.

Optionally, the bone cement mixing unit includes a bone cement extrusion assembly provided at one end inside the mixing housing, a return material extrusion assembly provided on an inner wall of the mixing housing and used in conjunction with the bone cement extrusion assembly, and a preliminary mixing assembly provided below the first olive-shaped chamber; and
the bone cement extrusion assembly includes a mounting member fixedly connected to a bottom wall of the mixing housing and a second motor fixedly connected to a side wall of one end of the mixing housing, one end of the mounting member is rotatably connected to a ball screw, one end of the ball screw passes through the mixing housing and is fixedly connected to an output shaft of a second motor, a first ball nut seat is spirally connected to the ball screw, a third motor is fixedly connected to a lower end of the first ball nut seat, an output shaft of the third motor passes through the first ball nut seat and is fixedly connected to a reciprocating screw, two second ball nut seats are symmetrically and spirally connected to upper and lower ends of the reciprocating screw, one end of each of the two second ball nut seats at the lower end is fixedly connected to a lower extrusion roller, and one end of each of the two second ball nut seats at the upper end is fixedly connected to an upper extrusion roller.

Optionally, the return material extrusion assembly includes a first motor fixedly connected to an outer side wall of the mixing housing, an output shaft of the first motor passes through the mixing housing and is fixedly connected to an indexing gear, a rectangular frame body is sleeved on an outer side of the indexing gear, extrusion racks are fixedly connected to upper and lower ends inside the rectangular frame body, the indexing gear is meshed with the two extrusion racks, one end of the rectangular frame body is fixedly connected to an L-shaped rod, one end of the L-shaped rod is fixedly connected to a storage extrusion portion, a limiting frame is fixedly connected to the inner wall of the mixing housing, the L-shaped rod passes through and is slidably connected to the limiting frame, another end of the rectangular frame body is fixedly connected to an ejector rod, and one end of the ejector rod is movably connected to a trigger assembly.

Optionally, the storage extrusion portion includes a cylinder fixedly connected to one end of the L-shaped rod, a horn-shaped storage port passes through the cylinder, and a large-end direction of the horn-shaped storage port faces the first olive-shaped chamber.

Optionally, the trigger assembly includes a mounting housing fixedly connected to the inner wall of the mixing housing, one side of the mounting housing passes through and is slidably connected to the ejector rod, one end of the ejector rod extending into the mounting housing is fixedly connected to a positive terminal, one end inside the mounting housing is fixedly connected to a cage, a negative terminal is fixedly connected to a middle of the cage, one end of the negative terminal is fixedly connected to a signal transceiving module, and the positive terminal, the negative terminal, and the signal transceiving module are connected in series.

Optionally, the preliminary mixing assembly includes two connecting seats fixedly connected to a middle of the bottom wall of the mixing housing, a rotating rod is rotatably connected between the two connecting seats, a V-shaped double-sided mixing paddle is fixedly connected to a middle of an outer circular surface of the rotating rod, the rotating rod passes through one of the connecting seats and is fixedly connected to an oscillating gear, an upper end of the oscillating gear is meshed with a reciprocating rack, unthreaded rods are fixedly connected to both ends of the reciprocating rack, two supports are further provided on the bottom wall of the mixing housing, the reciprocating rack, a combination of the unthreaded rods, and the two supports are all slidably connected, one end of one of the unthreaded rods is fixedly connected to an oscillating frame body, a fourth motor is further provided on the bottom wall of the mixing housing, an output shaft of the fourth motor is fixedly connected to an oscillating shaft connecting rod, one end of the oscillating shaft connecting rod is fixedly connected to an oscillating shaft, the oscillating shaft passes through the oscillating frame body, the reciprocating rack and the output shaft of the fourth motor are located on a same horizontal plane, and a length of the oscillating shaft connecting rod is half an axial length of the oscillating frame body.

Optionally, snap-fits are fixedly connected to four corners of one end of the mixing housing, slots are formed in four corners of one end of the extraction housing, and the extraction housing and the mixing housing are detachably connected through the snap-fits and the slots.

Compared with the prior art, the technical solutions provided by the present disclosure have at least the following beneficial effects:

In the above solutions, during vacuum mixing of bone cement raw materials, the device is compact and portable, allowing for its use directly within a narrow space, such as an operating room during an ongoing surgery. Moreover, by employing the bone cement mixing unit to perform uniform mixing and providing the storage battery for power supply, reducing manual labor intensity. Owing to the implementation of vacuum mixing, the device achieves a better mixing effect. Additionally, after the bone cement mixing is completed, the cleaning process requires only the detachment of the mixing housing, followed by rinsing the mixing chamber with clean water, without flushing the bone cement mixing unit that does not come into contact with the bone cement, thereby streamlining the cleaning procedure, and further improving the convenience in use.

The bone cement mixing unit pressurizes the bone cement within the first olive-shaped chamber, forcing it through the middle converging channel and into the second olive-shaped chamber. The middle converging channel itself acts as a mixing zone, and its small diameter ensures effective homogenization. The bone cement mixing unit then re-extrudes the bone cement that has been transferred into the second olive-shaped chamber, forcing it back through the middle converging channel and into the first olive-shaped chamber. This process is repeated. As the bone cement passes back and forth through the middle converging channel, it undergoes repeated pressurization and homogenization, thereby further improving the bone cement mixing effect.

Through the arrangement of the trigger assembly, when the rectangular frame body and its mounted components move to the leftmost position and are about to begin their return motion to the right, the ejector rod fixedly connected to the rectangular frame body actuates the contact between the positive terminal and the negative terminal. This activates the signal transceiving module. The signal transceiving module then notifies the control panel, which subsequently enables the bone cement extrusion assembly to initiate operation. Consequently, as the return material extrusion assembly returns to the right, the bone cement extrusion assembly simultaneously begins its own rightward motion for a new cycle, thereby enhancing the degree of automation of the device, and shortening the operational cycle time between the bone cement extrusion assembly and the return material extrusion assembly.

The preliminary mixing assembly is configured such that, prior to initiating the reciprocal extrusion and mixing by the bone cement extrusion assembly and the return material extrusion assembly, the control panel activates the fourth motor. This drives the oscillating shaft connecting rod and the oscillating shaft to rotate. As the oscillating shaft passes through the oscillating frame body, its rotation converts into a reciprocating linear motion of the oscillating frame body, the unthreaded rod, and the reciprocating rack. The linear motion of the reciprocating rack, in turn, drives the oscillating gear to rock back and forth. This oscillating gear motion drives the rotation of the rotating rod and the V-shaped double-sided mixing paddle, causing both sides of the paddle to alternately pat and mix the bottom of the first olive-shaped chamber, thereby achieving preliminary mixing of powder and liquid raw materials, facilitating the subsequent work of the bone cement extrusion assembly, and further enhancing the mixing effect.

### BRIEF DESCRIPTION OF THE DRAWINGS

The accompanying drawings incorporated herein and constituting a part of the specification show embodiments of the present disclosure. The accompanying drawings, together with the specification, are further used to explain the principles of the present disclosure, and enable those skilled in the relevant art to implement and use the present disclosure.

FIG. 1 is a three-dimensional structural diagram according to the present disclosure;

FIG. 2 is a structural diagram from another perspective of FIG. 1 according to the present disclosure;

FIG. 3 is a cross-sectional structural diagram of an extraction housing and components mounted thereon according to the present disclosure;

FIG. 4 is a cross-sectional structural diagram of a mixing housing and components mounted thereon according to the present disclosure;

FIG. 5 is a structural diagram of a bone cement mixing unit and a mixing chamber according to the present disclosure;

FIG. 6 is a structural diagram of a return material extrusion assembly according to the present disclosure;

FIG. 7 is a cross-sectional structural diagram of a storage extrusion portion in FIG. 6 according to the present disclosure;

FIG. 8 is a structural diagram of a bone cement extrusion assembly according to the present disclosure;

FIG. 9 is a cross-sectional structural diagram of a trigger assembly according to the present disclosure; and

FIG. 10 is a structural diagram of a preliminary mixing assembly according to the present disclosure.

### Reference Numerals

1. Extraction housing; 2. Mixing housing; 3. Liquid raw material extraction rod; 4. Control panel; 5. miniature vacuum pump;
6. Mixing chamber; 61. Trapezoidal interface chamber; 62. Connecting port; 63. First olive-shaped chamber; 64. Middle converging channel; 65. Second olive-shaped chamber;
7. Return material extrusion assembly; 71. First motor; 72. Indexing gear; 73. Rectangular frame body; 74. Extrusion rack; 75. L-shaped rod; 76. Limiting frame; 77. Storage extrusion portion; 771. Cylinder; 772. Horn-shaped storage port; 78. Ejector rod; 79. Trigger assembly; 791. Mounting housing; 792. Cage; 793. Signal transceiving module; 794. Negative terminal; 795. Positive terminal;
8. Bone cement extrusion assembly; 81. Mounting member; 82. Ball screw; 83. Second motor; 84. First ball nut seat; 85. Third motor; 86. Reciprocating screw; 87. Second ball nut seat; 88. Lower extrusion roller; 89. Upper extrusion roller;
9. Preliminary mixing assembly; 91. Connecting seat; 92. Rotating rod; 93. Oscillating gear; 94. V-shaped double-sided mixing paddle; 95. Support; 96. Reciprocating rack; 97. Unthreaded rod; 98. Oscillating frame body; 99. Oscillating shaft; 910. Oscillating shaft connecting rod; 911. Fourth motor;
10. Storage battery; 11. Powder raw material inlet; 12. Powder injection port; 13. Liquid raw material injection port; 14. Vacuum suction port; 15. Bone cement discharge port; 16. Snap-fit; 17. Slot; and 18. Liquid raw material storage cavity.

In order to clearly realize the structure of the embodiments of the present disclosure, specific structures and devices are marked in the figure. However, this is only for illustrative purposes and is not intended to limit the present disclosure to the specific structure, device and environment. According to specific needs, those of ordinary skill in the art can adjust or modify these devices and environments. However, the adjustments or modifications made are still included in the scope of the appended claims.

### DETAILED DESCRIPTION OF THE EMBODIMENTS

The present disclosure is described below in detail by using specific embodiments with reference to the accompanying drawings. Meanwhile, it needs to be explained here that in order to make the embodiments more detailed, the following embodiments are the best and preferred embodiments. For some well-known technologies, those skilled in the art can also implement them in other alternative ways. The drawings are only intended to describe the embodiments more specifically, rather than to define the present disclosure specifically.

It should be pointed out that “one embodiment”, “embodiments”, “exemplary embodiment”, “some embodiments” and other indicating words mentioned in the specification can include specific features, structures or characteristics, but not necessarily every embodiment includes the specific features, structures or characteristics. In addition, when describing specific features, structures or characteristics in combination with embodiments, the realization of such features, structures or characteristics in combination with other embodiments (whether explicitly described or not) is within the knowledge of those skilled in the relevant art.

In general, terms can be understood at least partially from their use in context. For example, depending at least in part on the context, the term “one or more” as used herein may be used to describe any feature, structure or characteristic in the singular sense, or a combination of features, structures or characteristics in the plural sense. In addition, the term “based on” can be understood as not necessarily intended to convey an exclusive set of factors, but alternatively, at least in part depending on the context, allowing for other factors that are not necessarily explicitly described.

It is understandable that the meanings of “on”, “over”, and “above” in the present disclosure should be interpreted in the broadest way. Thus, “on” means “directly on something”, and also means “on something and there is an intermediate feature or layer between”. “Over” and “above” mean “over and above something and there is no intermediate feature or layer between”.

In addition, for the convenience of description, spatial terms such as “below”, “under”, “lower part”, “above”, and “upper part” can be used herein to describe the relationship between one element or feature and another one or more elements or features, as shown in the attached drawings. These spatial terms are intended to cover different orientations in the use or operation of the device beyond those depicted in the drawings. Thus, a device may be oriented in another manner, and the spatial descriptors used herein may be similarly interpreted accordingly.

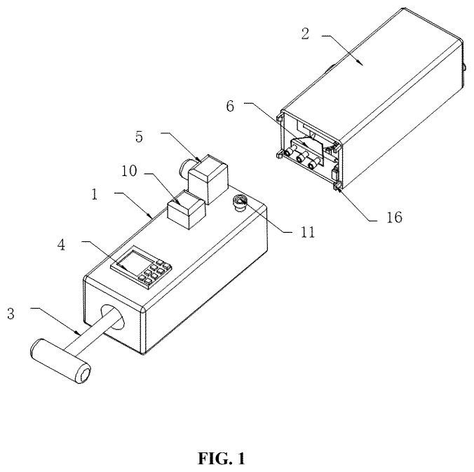

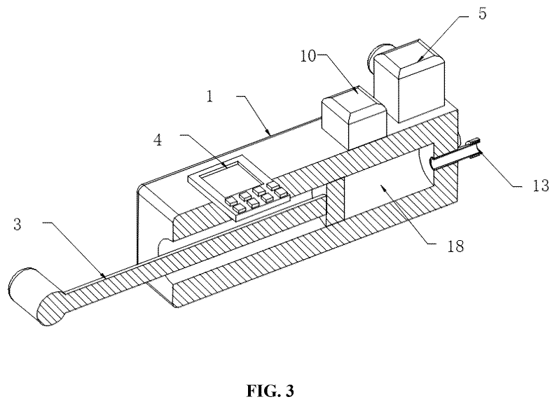

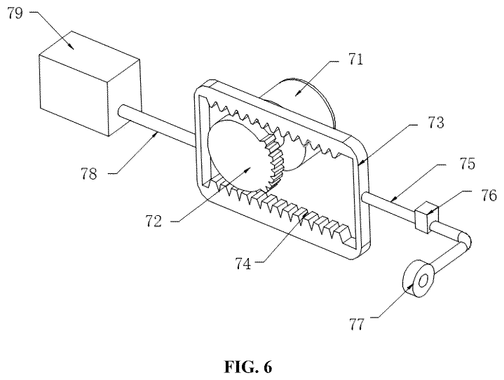

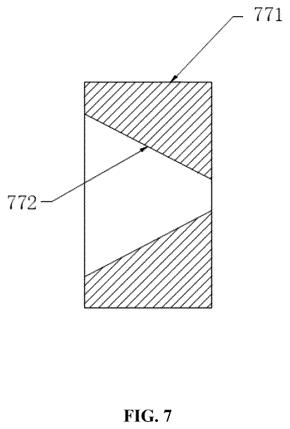

As shown in FIG. 1 to FIG. 10, embodiments of the present disclosure provide a portable vacuum mixing device for bone cement during surgery, including an extraction housing 1 and a mixing housing 2. The extraction housing 1 is detachably connected to the mixing housing 2. A miniature vacuum pump 5 is fixedly connected to an upper end of the extraction housing 1, a powder raw material inlet 11 is provided at a position of the upper end of the extraction housing 1 close to the miniature vacuum pump 5, a liquid raw material storage cavity 18 is formed inside the extraction housing 1, a liquid raw material injection port 13 communicating with the liquid raw material storage cavity 18 is formed in one end of the extraction housing 1, a liquid raw material extraction rod 3 is slidably and sealingly connected into the liquid raw material storage cavity 18, a powder injection port 12 and a vacuum suction port 14 are arranged symmetrically about the liquid raw material injection port 13 at one end of the extraction housing 1, the powder injection port 12 communicates with the powder raw material inlet 11 through a pipe, and the vacuum suction port 14 communicates with the miniature vacuum pump 5 through a pipe.

A mixing chamber 6 is provided inside the mixing housing 2, the mixing chamber 6 communicates with the liquid raw material injection port 13, the powder injection port 12, and the vacuum suction port 14, a bone cement discharge port 15 is formed in a middle of one end of the mixing housing 2, the bone cement discharge port 15 communicates with one end of the mixing chamber 6, and a bone cement mixing unit is provided inside the mixing housing 2.

A control panel 4 is provided on one side of an upper surface of the extraction housing 1, and a storage battery 10 is provided at a position of the upper surface of the extraction housing 1 close to the control panel 4.

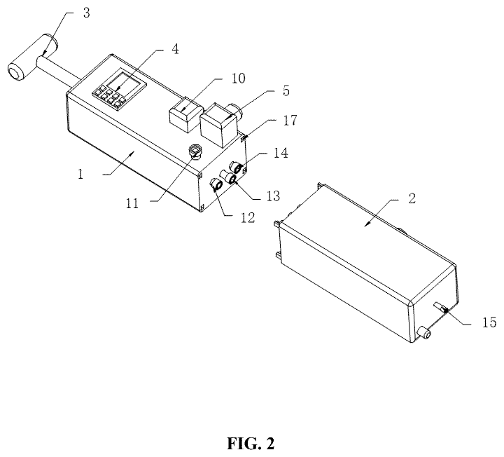

As shown in FIG. 1 and FIG. 2, snap-fits 16 are fixedly connected to four corners of one end of the mixing housing 2, slots 17 are formed in four corners of one end of the extraction housing 1, and the extraction housing 1 and the mixing housing 2 are detachably connected through the snap-fits 16 and the slots 17.

The device in the prior art cannot be employed in a confined space due to its overall bulk and poor portability, for instance, it cannot be deployed within an operating room during an ongoing surgical procedure. Furthermore, the reliance on manual mixing in the prior art not only imposes a substantial physical burden on the operator but also leads to suboptimal mixing outcomes. Additionally, the relatively complex mixing structure results in a cumbersome cleaning process after the bone cement has been mixed, indicating that there is still considerable room for improvement in terms of user convenience.

In the present disclosure, during use, the liquid raw material injection port 13 is first placed inside a storage device for a bone cement liquid raw material. It should be noted that the liquid raw material injection port 13 is longer than the powder injection port 12 and the vacuum suction port 14, facilitating the suction of the liquid raw material. Thereafter, the liquid raw material extraction rod 3 is manually pulled backwards (the liquid raw material extraction rod 3 is similar to a plunger rod, and a front end thereof is provided with a rubber plug slidably and sealingly connected to the liquid raw material storage cavity 18). After the liquid raw material extraction rod 3 is manually pulled, the volume of a liquid stored in the liquid raw material storage cavity 18 is increased to generate a negative pressure, suctioning the bone cement liquid raw material into the liquid raw material storage cavity 18, completing the preparation of the liquid raw material. The liquid raw material injection port 13 is then connected to one end of the mixing chamber 6 (the powder injection port 12, the liquid raw material injection port 13, and the vacuum suction port 14 are all detachably connected to the mixing chamber 6, and the detachable connection is optionally threaded connection, snap-fit connection, etc., which are not described in detail herein as the prior art). The powder injection port 12 and the vacuum suction port 14 are both fixed to and communicate with the mixing chamber 6. Finally, the extraction housing 1 and the mixing housing 2 are snap-fitted through the snap-fits 16 and the slots 17. In this way, the preparation of the device before mixing is completed.

The control panel 4 then controls the miniature vacuum pump 5 to operate, and the vacuum suction port 14 suctions air inside the mixing chamber 6, such that the mixing chamber 6 is internally vacuumized (before the mixed bone cement is discharged, solenoid valves provided inside the bone cement discharge port 15 are all in a closed state; the powder injection port 12, the liquid raw material injection port 13, and the vacuum suction port 14 are internally provided with solenoid valves, and when one of the three ports operates, the control panel 4 controls the solenoid valves inside the rest two ports to be closed; and in this state, the solenoid valves inside the powder injection port 12 and the liquid raw material injection port 13 are closed). After the vacuumizing operation is completed, the bone cement powder raw material is injected into the mixing chamber 6 through the powder raw material inlet 11 and the powder injection port 12, and in this case, the solenoid valves inside the liquid raw material injection port 13 and the vacuum suction port 14 are closed. It should be noted that when the bone cement powder raw material is injected through the powder raw material inlet 11 and the powder injection port 12, vacuum injection should be ensured. After the powder raw material is injected into the mixing chamber 6, the solenoid valve inside the liquid raw material injection port 13 is opened, the solenoid valves inside the powder injection port 12 and the vacuum suction port 14 are closed, and the liquid raw material extraction rod 3 is manually pressed to inject the liquid raw material into the mixing chamber 6 with the aid of the negative pressure inside the mixing chamber 6, completing the vacuum injection of the raw material. The solenoid valves inside the powder injection port 12, the liquid raw material injection port 13 and the vacuum suction port 14 are closed, and the bone cement mixing unit mixes the liquid raw material and the powder raw material inside the mixing chamber 6 to form bone cement paste. Finally, a finished product is discharged through the bone cement discharge port 15, completing the vacuum mixing operation of the bone cement.

In the present disclosure, during vacuum mixing of the bone cement raw materials, the device is compact and portable, allowing for its use directly within a narrow space, such as an operating room during an ongoing surgery. Moreover, by employing the bone cement mixing unit to perform uniform mixing and providing the storage battery 10 for power supply, reducing manual labor intensity. Owing to the implementation of vacuum mixing, the device achieves a better mixing effect. Additionally, after the bone cement mixing is completed, the cleaning process requires only the detachment of the mixing housing 2, followed by rinsing the mixing chamber 6 with clean water, without flushing the bone cement mixing unit that does not come into contact with the bone cement, thereby streamlining the cleaning procedure, and further improving the convenience in use.

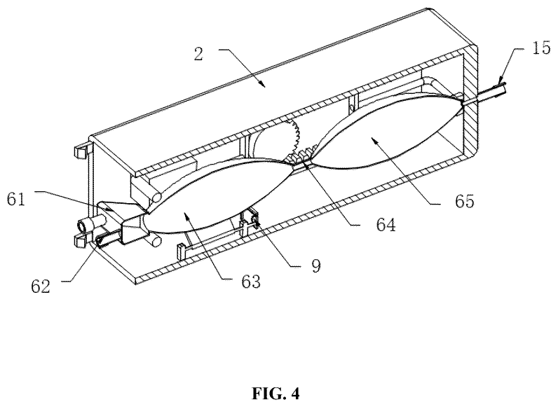

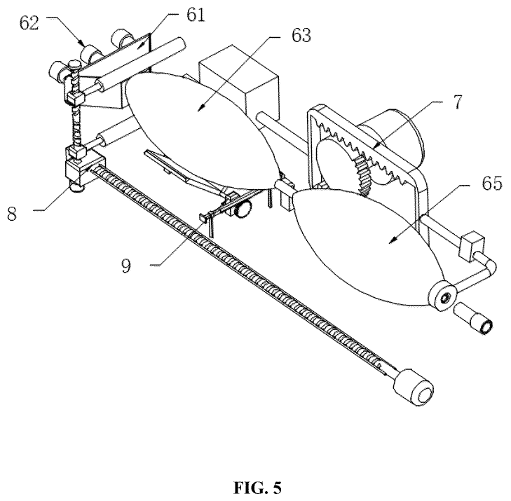

As shown in FIG. 4 and FIG. 5, the mixing chamber 6 includes a trapezoidal interface chamber 61, three connecting ports 62 are formed in one end of the trapezoidal interface chamber 61, the three connecting ports 62 are detachably connected to the powder raw material inlet 11, the powder injection port 12, and the vacuum suction port 14, respectively, one end of the trapezoidal interface chamber 61 is fixed to and communicates with a first olive-shaped chamber 63, one end of the first olive-shaped chamber 63 is fixed to and communicates with a middle converging channel 64, one end of the middle converging channel 64 is fixed to and communicates with a second olive-shaped chamber 65, and the second olive-shaped chamber 65 communicates with the bone cement discharge port 15.

The trapezoidal interface chamber 61, the first olive-shaped chamber 63, and the second olive-shaped chamber 65 are all made of rubber, and the middle converging channel 64 is made of hard plastic.

The mixing chamber 6 includes the trapezoidal interface chamber 61, the first olive-shaped chamber 63, the second olive-shaped chamber 65, and the middle converging channel 64. The powder raw material and the liquid raw material inside the mixing chamber 6 are injected through the powder injection port 12 and the liquid raw material injection port 13, first pass through the trapezoidal interface chamber 61 to enter the first olive-shaped chamber 63, and are pre-mixed inside the first olive-shaped chamber 63. After the mixing is completed, the bone cement mixing unit pressurizes the bone cement within the first olive-shaped chamber 63, forcing it through the middle converging channel 64 and into the second olive-shaped chamber 65. The middle converging channel 64 itself acts as a mixing zone, and its small diameter ensures effective homogenization. The bone cement mixing unit then re-extrudes the bone cement that has been transferred into the second olive-shaped chamber 65, forcing it back through the middle converging channel 64 and into the first olive-shaped chamber 63. This process is repeated. As the bone cement passes back and forth through the middle converging channel 64, it undergoes repeated pressurization and homogenization, thereby further improving the bone cement mixing effect.

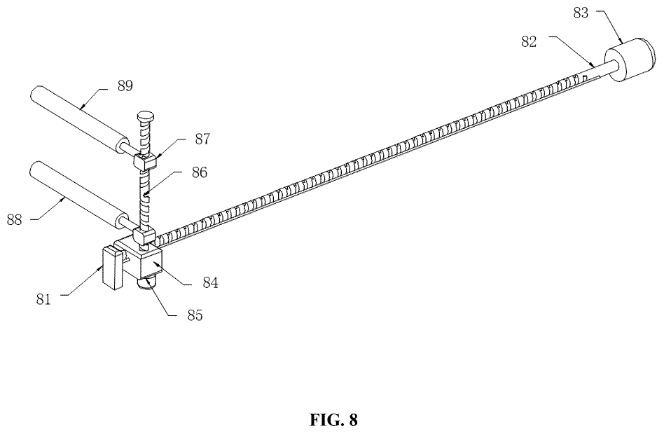

As shown in FIG. 5 and FIG. 8, the bone cement mixing unit includes a bone cement extrusion assembly 8 provided at one end inside the mixing housing 2, a return material extrusion assembly 7 provided on an inner wall of the mixing housing 2 and used in conjunction with the bone cement extrusion assembly 8, and a preliminary mixing assembly 9 provided below the first olive-shaped chamber 63.

The bone cement extrusion assembly 8 includes a mounting member 81 fixedly connected to a bottom wall of the mixing housing 2 and a second motor 83 fixedly connected to a side wall of one end of the mixing housing 2. One end of the mounting member 81 is rotatably connected to a ball screw 82, one end of the ball screw 82 passes through the mixing housing 2 and is fixedly connected to an output shaft of a second motor 83, a first ball nut seat 84 is spirally connected to the ball screw 82, a third motor 85 is fixedly connected to a lower end of the first ball nut seat 84, an output shaft of the third motor 85 passes through the first ball nut seat 84 and is fixedly connected to a reciprocating screw 86, two second ball nut seats 87 are symmetrically and spirally connected to upper and lower ends of the reciprocating screw 86, one end of each of the two second ball nut seats 87 at the lower end is fixedly connected to a lower extrusion roller 88, and one end of each of the two second ball nut seats 87 at the upper end is fixedly connected to an upper extrusion roller 89.

As shown in FIG. 5 to FIG. 7, the return material extrusion assembly 7 includes a first motor 71 fixedly connected to an outer side wall of the mixing housing 2. An output shaft of the first motor 71 passes through the mixing housing 2 and is fixedly connected to an indexing gear 72, a rectangular frame body 73 is sleeved on an outer side of the indexing gear 72, extrusion racks 74 are fixedly connected to upper and lower ends inside the rectangular frame body 73, the indexing gear 72 is meshed with the two extrusion racks 74, one end of the rectangular frame body 73 is fixedly connected to an L-shaped rod 75, one end of the L-shaped rod 75 is fixedly connected to a storage extrusion portion 77, a limiting frame 76 is fixedly connected to the inner wall of the mixing housing 2, and the L-shaped rod 75 passes through and is slidably connected to the limiting frame 76. Another end of the rectangular frame body 73 is fixedly connected to an ejector rod 78, and one end of the ejector rod 78 is movably connected to a trigger assembly 79.

The storage extrusion portion 77 includes a cylinder 771 fixedly connected to one end of the L-shaped rod 75, a horn-shaped storage port 772 passes through the cylinder 771, and a large-end direction of the horn-shaped storage port 772 faces the first olive-shaped chamber 63.

When the bone cement mixing unit operates, the bone cement extrusion assembly 8 first operates to extrude the bone cement inside the first olive-shaped chamber 63 into the second olive-shaped chamber 65 through the middle converging channel 64. Specifically, the control panel 4 controls the third motor 85 to operate, and the reciprocating screw 86 fixedly connected thereto is driven to rotate. When the reciprocating screw 86 rotates, the two second ball nut seats 87 at the upper and lower ends thereof oppositely move to the middle of the reciprocating screw 86, and drive the lower extrusion roller 88 and the upper extrusion roller 89 to approach mutually. After the lower extrusion roller 88 and the upper extrusion roller 89 abut against each other, the third motor 85 is turned off. The control panel 4 controls the second motor 83 to operate, and the second motor 83 can drive the fixedly connected ball screw 82 to rotate during rotation, such that the first ball nut seat 84 moves to the second motor 83 along the ball screw 82. As the first ball nut seat 84 moves, the lower extrusion roller 88 and the upper extrusion roller 89 are drive to move to the second motor 83, to extrude a raw material mixture inside the first olive-shaped chamber 63 to pass through the middle converging channel 64 and gradually enter the second olive-shaped chamber 65. When the lower extrusion roller 88 and the upper extrusion roller 89 move to the middle converging channel 64, the raw material mixture inside the first olive-shaped chamber 63 can be completely extruded into the second olive-shaped chamber 65. In this case, the second motor 83 is controlled to rotate reversely, to drive the lower extrusion roller 88 and the upper extrusion roller 89 to return to original positions (the positions shown in FIG. 5 being the original positions), completing primary mixing.

Next, the return material extrusion assembly 7 starts to operate, to extrude the raw material mixture inside the second olive-shaped chamber 65 into the first olive-shaped chamber 63 through the middle converging channel 64. Specifically, the control panel 4 controls the first motor 71 to operate, to drive the indexing gear 72 to rotate anticlockwise, and the indexing gear 72 drives the rectangular frame body 73 and the components mounted thereon to move leftwards (the direction shown in FIG. 5) together when being meshed with the extrusion rack 74 at the upper end of the rectangular frame body 73, such that the storage extrusion portion 77 converges and extrudes the second olive-shaped chamber 65, and the raw material mixture inside the second olive-shaped chamber 65 is re-extruded into the first olive-shaped chamber 63 through the middle converging channel 64 for secondary mixing. The horn-shaped storage port 772 passes through the cylinder 771, and the large-end direction of the horn-shaped storage port 772 faces the first olive-shaped chamber 63, facilitating the extrusion operation. It should be noted that when the rectangular frame body 73 and the components mounted thereon move to the leftmost side together, the storage extrusion portion 77 exactly moves to the middle converging channel 64, such that the raw material mixture inside the second olive-shaped chamber 65 can be completely extruded into the first olive-shaped chamber 63. When the indexing gear 72 continuously moves anticlockwise to mesh with the lower extrusion roller 74, the rectangular frame body 73 and the components mounted thereon are driven to move rightwards together to reset (the positions shown in FIG. 5 indicating an initial state).

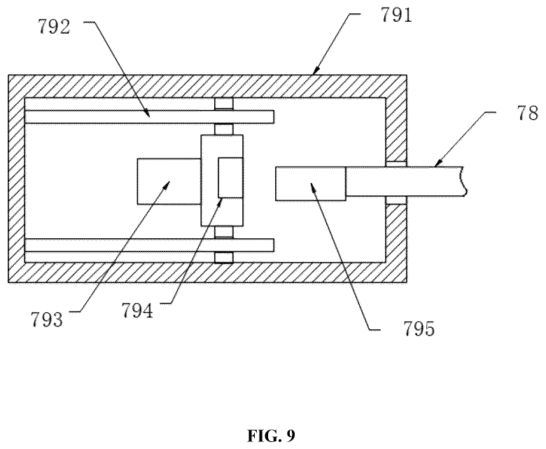

As shown in FIG. 9, the trigger assembly 79 includes a mounting housing 791 fixedly connected to the inner wall of the mixing housing 2. One side of the mounting housing 791 passes through and is slidably connected to the ejector rod 78, one end of the ejector rod 78 extending into the mounting housing 791 is fixedly connected to a positive terminal 795, one end inside the mounting housing 791 is fixedly connected to a cage 792, a negative terminal 794 is fixedly connected to a middle of the cage 792, one end of the negative terminal 794 is fixedly connected to a signal transceiving module 793, and the positive terminal 795, the negative terminal 794, and the signal transceiving module 793 are connected in series.

During the operation of the return material extrusion assembly 7, when the rectangular frame body 73 and components mounted thereon move to the leftmost position and are about to begin their return motion to the right, the ejector rod 78 fixedly connected to the rectangular frame body 73 actuates the contact between the positive terminal 795 and the negative terminal 794. This activates the signal transceiving module 793. The signal transceiving module 793 then notifies the control panel 4, which subsequently enables the bone cement extrusion assembly 8 to initiate operation. Consequently, as the return material extrusion assembly 7 returns to the right, the bone cement extrusion assembly 8 simultaneously begins its own rightward motion for a new cycle, thereby enhancing the degree of automation of the device, and shortening the operational cycle time between the bone cement extrusion assembly 8 and the return material extrusion assembly 7.

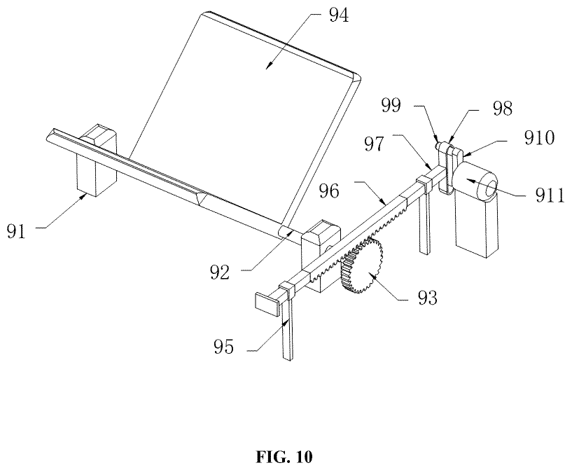

As shown in FIG. 10, the preliminary mixing assembly 9 includes two connecting seats 91 fixedly connected to a middle of the bottom wall of the mixing housing 2. A rotating rod 92 is rotatably connected between the two connecting seats 91, a V-shaped double-sided mixing paddle 94 is fixedly connected to a middle of an outer circular surface of the rotating rod 92, the rotating rod 92 passes through one of the connecting seats 91 and is fixedly connected to an oscillating gear 93, an upper end of the oscillating gear 93 is meshed with a reciprocating rack 96, unthreaded rods 97 are fixedly connected to both ends of the reciprocating rack 96, two supports 95 are further provided on the bottom wall of the mixing housing 2, the reciprocating rack 96, a combination of the unthreaded rods 97, and the two supports 95 are all slidably connected, one end of one of the unthreaded rods 97 is fixedly connected to an oscillating frame body 98, a fourth motor 911 is further provided on the bottom wall of the mixing housing 2, an output shaft of the fourth motor 911 is fixedly connected to an oscillating shaft connecting rod 910, one end of the oscillating shaft connecting rod 910 is fixedly connected to an oscillating shaft 99, the oscillating shaft 99 passes through the oscillating frame body 98, the reciprocating rack 96 and the output shaft of the fourth motor 911 are located on a same horizontal plane, and a length of the oscillating shaft connecting rod 910 is half an axial length of the oscillating frame body 98.

When the liquid raw material and the powder raw material are injected into the first olive-shaped chamber 63 for the first time, there is an obvious layering phenomenon due to separate injection of the two. In this case, it is difficult to extrude the liquid raw material and the powder raw material in the first olive-shaped chamber 63 into the second olive-shaped chamber 65 by the bone cement extrusion assembly 8. The preliminary mixing assembly 9 is configured such that, prior to initiating the reciprocal extrusion and mixing by the bone cement extrusion assembly 8 and the return material extrusion assembly 7, the control panel 4 activates the fourth motor 911. This drives the oscillating shaft connecting rod 910 and the oscillating shaft 99 to rotate. As the oscillating shaft 99 passes through the oscillating frame body 98, its rotation converts into a reciprocating linear motion of the oscillating frame body 98, the unthreaded rod 97, and the reciprocating rack 96. The linear motion of the reciprocating rack 96, in turn, drives the oscillating gear 93 to rock back and forth. This oscillating gear motion drives the rotation of the rotating rod 92 and the V-shaped double-sided mixing paddle 94, causing both sides of the paddle to alternately pat and mix the bottom of the first olive-shaped chamber 63 (the first olive-shaped chamber 63 is made of rubber and has certain elasticity; and after being injected, the powder and liquid raw materials are deposited at the lower half part of the first olive-shaped chamber 63 under the action of gravity), thereby achieving preliminary mixing of the powder and liquid raw materials, facilitating the subsequent work of the bone cement extrusion assembly 8, and further enhancing the mixing effect.

The present disclosure provides the following working process in the technical solutions:

In the present disclosure, during use, the liquid raw material injection port 13 is first placed inside a storage device for a bone cement liquid raw material. It should be noted that the liquid raw material injection port 13 is longer than the powder injection port 12 and the vacuum suction port 14, facilitating the suction of the liquid raw material. Thereafter, the liquid raw material extraction rod 3 is manually pulled backwards (the liquid raw material extraction rod 3 is similar to a plunger rod, and a front end thereof is provided with a rubber plug slidably and sealingly connected to the liquid raw material storage cavity 18). After the liquid raw material extraction rod 3 is manually pulled, the volume of a liquid stored in the liquid raw material storage cavity 18 is increased to generate a negative pressure, suctioning the bone cement liquid raw material into the liquid raw material storage cavity 18, completing the preparation of the liquid raw material. The liquid raw material injection port 13 is then connected to one end of the mixing chamber 6 (the powder injection port 12, the liquid raw material injection port 13, and the vacuum suction port 14 are all detachably connected to the mixing chamber 6, and the detachable connection is optionally threaded connection, snap-fit connection, etc., which are not described in detail herein as the prior art). The powder injection port 12 and the vacuum suction port 14 are both fixed to and communicate with the mixing chamber 6. Finally, the extraction housing 1 and the mixing housing 2 are snap-fitted through the snap-fits 16 and the slots 17. In this way, the preparation of the device before mixing is completed.

The control panel 4 then controls the miniature vacuum pump 5 to operate, and the vacuum suction port 14 suctions air inside the mixing chamber 6, such that the mixing chamber 6 is internally vacuumized (before the mixed bone cement is discharged, solenoid valves provided inside the bone cement discharge port 15 are all in a closed state; the powder injection port 12, the liquid raw material injection port 13, and the vacuum suction port 14 are internally provided with solenoid valves, and when one of the three ports operates, the control panel 4 controls the solenoid valves inside the rest two ports to be closed; and in this state, the solenoid valves inside the powder injection port 12 and the liquid raw material injection port 13 are closed). After the vacuumizing operation is completed, the bone cement powder raw material is injected into the mixing chamber 6 through the powder raw material inlet 11 and the powder injection port 12, and in this case, the solenoid valves inside the liquid raw material injection port 13 and the vacuum suction port 14 are closed. It should be noted that when the bone cement powder raw material is injected through the powder raw material inlet 11 and the powder injection port 12, vacuum injection should be ensured. After the powder raw material is injected into the mixing chamber 6, the solenoid valve inside the liquid raw material injection port 13 is opened, the solenoid valves inside the powder injection port 12 and the vacuum suction port 14 are closed, and the liquid raw material extraction rod 3 is manually pressed to inject the liquid raw material into the mixing chamber 6 with the aid of the negative pressure inside the mixing chamber 6, completing the vacuum injection of the raw material. The solenoid valves inside the powder injection port 12, the liquid raw material injection port 13 and the vacuum suction port 14 are closed, and the bone cement mixing unit mixes the liquid raw material and the powder raw material inside the mixing chamber 6 to form bone cement paste.

Upon completion of the mixing of the bone cement, it is only necessary to activate the bone cement extrusion assembly 8, such that the lower extrusion roller 88 and the upper extrusion roller 89 move to the second olive-shaped chamber 65 on the rightmost side, thereby extruding the finished bone cement through the bone cement discharge port 15.

The present disclosure covers any substitution, modification, equivalent method and solution made within the spirit and scope of the present disclosure. For a better understanding of the present disclosure, the specific details of the following preferred embodiments of the present disclosure are explained hereinafter in detail, while the present disclosure can also be fully understood by those skilled in the art without the description of these details. In addition, in order to avoid unnecessary confusion of the essence of the present disclosure, well-known methods, processes, flowcharts, elements, and circuits are not described in detail.

The foregoing description is only preferred implementation of the present disclosure. It should be noted that a person of ordinary skill in the art can also make several improvements and modifications without departing from the principle of the present disclosure. These improvements and modifications should also be deemed as falling within the protection scope of the present disclosure.

What is claimed is:

1. A portable vacuum mixing device for bone cement during surgery, comprising:
an extraction housing and a mixing housing, wherein the extraction housing is detachably connected to the mixing housing;
a miniature vacuum pump is fixedly connected to an upper end of the extraction housing, a powder raw material inlet is provided at a position of the upper end of the extraction housing close to the miniature vacuum pump, a liquid raw material storage cavity is formed inside the extraction housing, a liquid raw material injection port communicating with the liquid raw material storage cavity is formed in one end of the extraction housing, a liquid raw material extraction rod is slidably and sealingly connected into the liquid raw material storage cavity, a powder injection port and a vacuum suction port are arranged symmetrically about the liquid raw material injection port at one end of the extraction housing, the powder injection port communicates with the powder raw material inlet through a pipe, and the vacuum suction port communicates with the miniature vacuum pump through a pipe; and
a mixing chamber is provided inside the mixing housing, the mixing chamber communicates with the liquid raw material injection port, the powder injection port, and the vacuum suction port, a bone cement discharge port is formed in a middle of one end of the mixing housing, the bone cement discharge port communicates with one end of the mixing chamber, and a bone cement mixing unit is provided inside the mixing housing;
wherein the bone cement mixing unit comprises a bone cement extrusion assembly provided at one end inside the mixing housing, a return material extrusion assembly provided on an inner wall of the mixing housing and used in conjunction with the bone cement extrusion assembly, and a preliminary mixing assembly provided below the first olive-shaped chamber; and
the bone cement extrusion assembly comprises a mounting member fixedly connected to a bottom wall of the mixing housing and a second motor fixedly connected to a side wall of one end of the mixing housing, one end of the mounting member is rotatably connected to a ball screw, one end of the ball screw passes through the mixing housing and is fixedly connected to an output shaft of a second motor, a first ball nut seat is spirally connected to the ball screw, a third motor is fixedly connected to a lower end of the first ball nut seat, an output shaft of the third motor passes through the first ball nut seat and is fixedly connected to a reciprocating screw, two second ball nut seats are symmetrically and spirally connected to upper and lower ends of the reciprocating screw, one end of each of the two second ball nut seats at the lower end of the reciprocating screw is fixedly connected to a lower extrusion roller, and one end of each of the two second ball nut seats at the upper end of the reciprocating screw is fixedly connected to an upper extrusion roller;
wherein the preliminary mixing assembly comprises two connecting seats fixedly connected to a middle of the bottom wall of the mixing housing, a rotating rod is rotatably connected between the two connecting seats, a V-shaped double-sided mixing paddle is fixedly connected to a middle of an outer circular surface of the rotating rod, the rotating rod passes through one of the two connecting seats and is fixedly connected to an oscillating gear, an upper end of the oscillating gear is meshed with a reciprocating rack, unthreaded rods are fixedly connected to both ends of the reciprocating rack, two supports are further provided on the bottom wall of the mixing housing, the reciprocating rack, a combination of the unthreaded rods, and the two supports are all slidably connected, one end of one of the unthreaded rods is fixedly connected to an oscillating frame body, a fourth motor is further provided on the bottom wall of the mixing housing, an output shaft of the fourth motor is fixedly connected to an oscillating shaft connecting rod, one end of the oscillating shaft connecting rod is fixedly connected to an oscillating shaft, the oscillating shaft passes through the oscillating frame body, the reciprocating rack and the output shaft of the fourth motor are located on a same horizontal plane, and a length of the oscillating shaft connecting rod is half an axial length of the oscillating frame body.

2. The portable vacuum mixing device for bone cement during surgery according to claim 1, wherein a control panel is provided on one side of an upper surface of the extraction housing, and a storage battery is provided at a position of the upper surface of the extraction housing close to the control panel.

3. The portable vacuum mixing device for bone cement during surgery according to claim 1, wherein the mixing chamber comprises a trapezoidal interface chamber, three connecting ports are formed in one end of the trapezoidal interface chamber, the three connecting ports are detachably connected to the powder raw material inlet, the powder injection port, and the vacuum suction port, respectively, one end of the trapezoidal interface chamber is fixed to and communicates with a first olive-shaped chamber, one end of the first olive-shaped chamber is fixed to and communicates with a middle converging channel, one end of the middle converging channel is fixed to and communicates with a second olive-shaped chamber, and the second olive-shaped chamber communicates with the bone cement discharge port.

4. The portable vacuum mixing device for bone cement during surgery according to claim 3, wherein the trapezoidal interface chamber, the first olive-shaped chamber, and the second olive-shaped chamber are all made of rubber, and the middle converging channel is made of hard plastic.

5. The portable vacuum mixing device for bone cement during surgery according to claim 1, wherein the return material extrusion assembly comprises a first motor fixedly connected to an outer side wall of the mixing housing, an output shaft of the first motor passes through the mixing housing and is fixedly connected to an indexing gear, a rectangular frame body is sleeved on an outer side of the indexing gear, two extrusion racks are fixedly connected to upper and lower ends inside the rectangular frame body, respectively, the indexing gear is meshed with the two extrusion racks, one end of the rectangular frame body is fixedly connected to an L-shaped rod, one end of the L-shaped rod is fixedly connected to a storage extrusion portion, a limiting frame is fixedly connected to the inner wall of the mixing housing, the L-shaped rod passes through and is slidably connected to the limiting frame, another end of the rectangular frame body is fixedly connected to an ejector rod, and one end of the ejector rod is movably connected to a trigger assembly.

6. The portable vacuum mixing device for bone cement during surgery according to claim 5, wherein the storage extrusion portion comprises a cylinder fixedly connected to one end of the L-shaped rod, a horn-shaped storage port passes through the cylinder, and a large-end direction of the horn-shaped storage port faces the first olive-shaped chamber.

7. The portable vacuum mixing device for bone cement during surgery according to claim 5, wherein the trigger assembly comprises a mounting housing fixedly connected to the inner wall of the mixing housing, one side of the mounting housing passes through and is slidably connected to the ejector rod, one end of the ejector rod extending into the mounting housing is fixedly connected to a positive terminal, one end inside the mounting housing is fixedly connected to a cage, a negative terminal is fixedly connected to a middle of the cage, one end of the negative terminal is fixedly connected to a signal transceiving module, and the positive terminal, the negative terminal, and the signal transceiving module are connected in series.

8. The portable vacuum mixing device for bone cement during surgery according to claim 1, wherein snap-fits are fixedly connected to four corners of one end of the mixing housing, slots are formed in four corners of one end of the extraction housing, and the extraction housing and the mixing housing are detachably connected through the snap-fits and the slots.
